(19) United States
(12) Patent Application Publication
(10) Pub. No.: US 2004/0022813 A1
(43) Pub. Date: Feb. 5, 2004

(54) Shed antigen vaccine with dendritic cells adjuvant

(76) Inventor: Jean-Claude Bystryn, New York, NY (US)

Correspondence Address: Robert D. Katz, Cooper & Dunham LLP, 1185 Avenue of the Americas, New York, NY, 10036, US

(21) Appl. No.: 10/213,388

(22) Filed: Aug. 5, 2002

(51) Int. Cl. A61K 39/00

(52) U.S. Cl. 424/277.1

(57) ABSTRACT

The invention provides a method for producing a composition for use as a vaccine for treatment or prevention of cancer, comprising collecting antigens released or shed by the type of tumor cell against which it is desired to prepare the vaccine; preparing mammalian dendritic cells in a culture from a mammalian blood, bone marrow or other tissue sample by culturing the blood, bone marrow, or other tissue sample under conditions that cause differentiation and proliferation of dendritic cells; separating dendritic cells from other cells in the culture; and exposing the dendritic cells to the shed antigens collected as described in paragraph a. above under conditions that result in the combination of the shed cancer antigens or their fragments and the dendritic cells. The invention also provides compositions for administration as a vaccine for the treatment of cancer, and other diseases.

### FIELD OF THE INVENTION

This invention relates to shed antigen vaccines for the treatment of human melanoma, breast cancer and other cancers, and more particularly to a human cancer vaccine having an improved adjuvant derived from, or including, dendritic cells or other types of antigen presenting cells, which present the shed tumor antigens to T-cells in order to stimulate an anti-tumor immune response in a patient afflicted with such a disease. This invention can also be applied to prepare improved vaccines against infectious and autoimmune diseases.

### BACKGROUND OF THE INVENTION

Various treatments for cancer exist, including surgery, which physically removes cancerous tissue, radiation, which seeks to kill cancer cells, and chemotherapy, which also targets more rapidly proliferating cells in a person affected with cancer.

There also exists a variety of treatments that seek to more selectively destroy the cancer cells by provoking an immune response against the cancerous cells, without attacking healthy cells, by using cancer vaccines. This category includes a number of different vaccine approaches, which all include administering one or more antigens associated with the cancer in order to provoke an immune response against the tumor or cancer cells, and seeks to cause tumor shrinkage or remission. The types and sources of antigens administered, as well as the method of administration differ among the various approaches.

The most critical factors in constructing cancer vaccines, and vaccines for other diseases, are the selection of the antigens used to prepare the vaccine and the procedure or adjuvant that is combined with the vaccine to increase the strength of the immune responses that are induced by the vaccine. The invention herein describes a procedure to construct improved cancer vaccines and vaccines for other diseases based on combining a particularly effective antigen preparation with a particularly effective way of enhancing the immune responses stimulated by these antigens.

Cancer vaccines are intended to stimulate immune responses against cancer cells and by so doing, increase a patient's resistance to the cancer and slow or prevent its progression. Similar principles apply to vaccines intended to treat or prevent infectious or autoimmune diseases.

The rationale for believing that cancer vaccines can work has been reviewed (Bystryn, J-C, et al., “Clinical applications: Partially Purified Tumor Antigen Vaccines,” Biol. Ther. of Cancer, 2nd Ed., ed. V. DeVita, S. Hellman, and S. A. Rosenberg, J. B. Lippincott, Philadelphia, Pa., pp. 669-69, 1995)¹. The most convincing evidence that they can be effective is that they can prevent cancer in animals. For example, melanoma vaccine-immunized mice survive challenge with a lethal number of melanoma cells that invariably kills all non-immunized mice (Bystryn, J-C, “Antibody Response and Tumor Growth in Syngeneic Mice Immunized to Partially Purified B16 Melanoma Associated Antigens,” J. Immunol. 120:96-101, 1978). The results of initial clinical trials of some cancer vaccines in humans are promising, as evidenced by regression or delayed progression of established metastases and by prolongation of disease-free and overall survival in patients with resected disease (Morton, D. L. et al., ¹The patents, patent applications, and other references cited in this application are incorporated herein by reference. C A Caner: J. Clin., 46:225-244, 1996; Berd, D., et al., “Autologous Hapten-modified Melanoma Vaccine as Postsurgical Adjuvant Treatment After Resection of Nodal Metastases,” J. Clin. Oncol., 15:2359-2370,1997; Rosenberg, S. A., et al., “Immunologic an Therapeutic Evaluation of a Synthetic Peptide Vaccine for the Treatment of Patients with Metastatic Melanoma,” Nat. Med., 4:321-327, 1998; Nestle, F. O., et al., “Vaccination of Melanoma Patients with Peptide or Tumor Lysate-pulsed Dendritic Cells,” Nat. Med., 4: 328-332, 1998; and Mitchell, M. S., “Perspective on Allogenic Melanoma Lysates in Active Specific Immunotherapy,” Semin. Oncol., 25: 623-635, 1998). As an example, in our studies, the median recurrence-free and overall survival of patients with resected AJCC stage III melanoma treated with a polyvalent shed antigen melanoma vaccine was approximately twice as long as that of similar, historical, control patients (Bystryn, J-C, et al., “Clinical applications: Partially Purified Tumor Antigen Vaccines,” 1995). More convincingly, in a double-blind and placebo controlled trial, the recurrence-free survival of melanoma vaccine treated patients was two and half fold longer than that of similar patients treated with a placebo vaccine, and this difference was statistically significant after Cox multi variate analysis, p=0.03 (Bystryn, J-C et al., “Double-Blind Trial of a Polyvalent, Shed-Antigen, Melanoma Vaccine”, Clin. Cancer Res. 7:1882-1887, 2001).

The beneficial effects of vaccine treatment are mediated by stimulation of antitumor immune responses. This is evidenced in animals by the specificity of vaccine-induced tumor protective effects. As an example, mice immunized to a murine B16 melanoma vaccine are not protected against challenge by an unrelated syngeneic murine tumor, while mice immunized to a control vaccine are not protected against B16 melanoma (Bystryn, J-C, “Antibody Response and Tumor Growth in Syngeneic Mice Immunized to Partially Purified B16 Melanoma Associated Antigens,” J. Immunol. 120: 96-101, 1978). It is evidenced in man by correlations between vaccine-induced antitumorcellular (Reynolds, S. R., et al., “Stimulation of CD8+T Cell Responses to MAGE-3 and MELAN A/MART-1 By Immunization to a Polyvalent Melanoma Vaccine,” Int. J. Cancer, 72:972-502,1995; and 14) or antibody (Miller, K. et al., “Improved Survival of Melanoma Patients with an Antibody Response to Immunization to a Polyvalent Melanoma Vaccine,” Cancer, 75(2):495-502, 1995; Takahashi, T., et al., IgM antiganglioside antibodies induced by melanoma cell vaccine correlate with survival of Melanoma Patients, J. Invest. Dermato., 112:205-09,1999; and Livingston, P. O., et al., “Improved Survival in Stage III Melanoma Patients with GM2 Antibodies,” J. Clin. Oncol. 12:1036-1044, 1994) responses and improved clinical outcome. The implication of these observations is that the clinical effectiveness of cancer vaccines depends on their ability to stimulate anti-tumor immune responses.

One of the most critical elements in the preparation of an effective vaccine against cancer is the antigens used to construct the vaccine. These must be able to trigger clinically effective immune responses in humans that can attack and destroy tumor cells. Furthermore, some of these responses must be directed against antigens present on the external surface of the patient's own tumor where they can be seen and attacked by the immune responses.

A variety of procedures are currently used to obtain tumor antigens for cancer vaccines. One approach includes administering to a patient killed whole tumor cells or a lysate of tumor cells of the particular cancer involved as the antigen. The cells or lysate may come from an established cancer cell line, prepared by conventional techniques such as repetitively freezing and thawing the cell sample. Alternatively, a lysate of the surgical sample of the cancer from the particular patient being treated may provide the lysate for use as an antigen. Other antigen preparations include a membrane preparation from a tumor, either from a cell line or a specimen from the patient. Likewise, antigenic purified amino acid sequences characteristic of the tumor cell have been employed as cancer antigens. Various types of antigens asserted to be useful in tumor vaccines are discussed in U.S. Pat. Nos. 5,788,963 and 6,017,527, both of which are incorporated by reference herein.

Such antigens, including tumor antigens, in many cases have failed to live up to their promise. Many vaccines for treatment of melanoma and other cancers have had disappointing clinical results, while others are too weak or have too many side effects. The probable reasons that cancer vaccines may not be effective are that the vaccine fails to induce immune responses against the patients own cancer cells, and the responses which are induced are not sufficiently potent to destroy the tumor cells. Thus the critical need to construct vaccines from relevant antigens and to combine these antigens with a procedure that will strongly augment the immune responses induced by these antigens.

Two problems make it difficult to select antigens that are appropriate to construct cancervaccines. One is that the identity of the individual tumor antigens that can trigger clinically effective anti-tumor immune response in humans remains mostly unknown. While many antigens associated with various human tumors have been identified, and a few that can trigger immune responses in humans have also been identified, little is known about which if any of these antigens triggers the type of immune responses that will kill tumor cells in vivo. We know that such antigens are expressed by tumors, but we don't know which of the many antigens on a tumor are the desired ones. The other problem is that tumor cells are antigenically heterogeneous. This means that the individual tumor antigens expressed by tumor cells varies from individual to individual, between different tumor nodules in the same individual, and in fact within the same tumor nodule. Furthermore, the actual tumor antigens expressed by a patient's own tumor are usually not known (as these are difficult to measure and in many cases the tumor has already been removed by the time this information is sought); and even if known, the individuals' antigens expressed can change during the natural progression of the cancer. Thus, we do not know what individual tumor antigens should be used to prepare a vaccine, and we do not know which if any of the antigens that are needed will be present on the particular tumor that needs to be treated.

Rationale for preparing polyvalent cancer vaccines from shed antigens: One approach to overcome the problems described above is to prepare polyvalent vaccines that contain numerous tumor-associated antigens from antigens which are shed into culture medium by tumor cells, as disclosed in U.S. Pat. No. 6,338,853 (Bystryn). The advantages of this approach are multiple. First, polyvalent vaccines that contain multiple tumor antigens are desirable since the greater the number of antigens in the vaccine: a) the greater the chance that the vaccine will contain those still unknown antigens that stimulate tumor protective immunity and obviate the need to identify and purify the individual tumor antigens that do so; b) the greater the chance that the vaccine will contain antigens present on the tumor to be treated, and thus circumvent the antigenic heterogeneity of tumor cells; c) the greater the chance that the vaccine will be able to circumvent HLA dependent and independent heterogeneity in the ability of different individuals to develop immune responses to any particular antigen (Reynolds et al., “HLA-Independent Heterogeneity of CD8+ T Cell Responses to MAGE-3, Melan A/MART-1, gp100, Tyrosinase, MC1R and TRP-2 in Vaccine-Treated Melanoma Patients,” J. Immunol., 161: 6970-6976,1998); and d) the less chance that the tumor will escape from immune recognition, stimulation of immune responses to multiple targets on tumor cells will increase the chances of tumor destruction. This seems intuitive, since if immune responses against one antigenic target can damage a tumor cell, responses directed against multiple targets should cause even more damage.

We have developed a unique approach to prepare polyvalent vaccines that we believe has significant advantages over alternate procedures to make cancer vaccines. It is to prepare the vaccine from tumor-associated antigens that are released (shed) from the surface of tumor cells into their culture medium. The rationale for this approach has been published (Bystryn, J-C et al., “Cancer Vaccines: Clinical Applications: Partially Purified Tumor Antigen Vaccines,” in Biologic Therapy of Cancer, 2^{nd} Edition, ed. by V. DeVita, S. Hellman and S. A. Rosenberg; J. B. Lippincott: Philadelphia, pp 668-679, 1995), and several patents have been issued on the procedure. Briefly, tumor cells rapidly release or “shed” into culture medium a broad range of molecules, including tumor antigens, expressed on their external surface. Release can be enhanced by treating the cells at an acidic pH, with enzymes or other agents that strip off surface material. The shed material provides a unique source of material from which to construct cancer vaccines, including a rich source of multiple tumor antigens, as a large proportion of the material present on the external surface of the cells is released without a few hours. The spectrum of tumor-associated antigens can be further increased by collecting and pooling the material shed by several tumor cell lines, selected because they express different and complimentary patterns of tumor antigens. Shed antigens are more likely to be biologically relevant for vaccine immunotherapy than antigens present inside the cells, as they are expressed on the external surface of tumor cells, where they can be seen and attacked by vaccine-induced anti-tumor immune responses. Shed antigens are highly purified, as they are separated from the bulk of cellular material which is in the cytoplasm and nucleus and is poorly shed. This is in contrast to polyvalent vaccine prepared from whole tumor cells or their lysate, as the overwhelming bulk of material and antigens in such vaccines is cytoplasmic and nuclear material.

By contrast, the usual methods of preparing polyvalent vaccines is to make them from tumor cells or their lysate or by mixing several purified antigens. Compared to vaccines prepared from whole tumor cells or their lysate, vaccines made from shed antigens are much purer as they are separated from the bulk of the cellular material which is in the cytoplasm and the nucleus of cells and is poorly shed. Furthermore, the concentration of relevant tumor antigens, which are those present on the external surface of the tumor cells, is much greater and that of potentially dangerous material inside the cells is reduced. In contrast to vaccines made from several purified tumor antigens, vaccines made from shed antigens contain a much greater range of tumor-associated antigens.

This vaccine has provided satisfactory results in clinical trials with melanoma patients, including a statistically significant prolongation of recurrence-free survival in a double-blind and placebo controlled trial in patients with resected melanoma. However, the vaccine can benefit from an improved adjuvant, which may increase its effectiveness.

The need for adjuvants to increase the potency of vaccines: Unfortunately, most cancer vaccines are poorly immunogenic. They often fail to stimulate anti-tumor immune responses and the responses which are induced can be infrequent, weak and of a short duration. The same is true for some vaccines against infections diseases or potential vaccines for autoimmune diseases. Consequently, a major challenge in the design of all types of vaccines is to develop immunization procedures that will boost vaccine immunogenicity. A broad range of different adjuvants has been developed to address this problem. This includes various types of oils, mineral salts such as alum, bacterial extracts, cytokines, beads and other types of particles. Unfortunately, many of these fail to enhance sufficiently the effectiveness of vaccines.

The procedure which appears to be one of the most effective to enhance vaccine induced immune responses is to combine the antigens in the vaccine with dendritic or other types of antigen presenting cells. These are specialized cells whose function it is to capture antigens and present them to other types of immune cells in order to trigger immune responses.

Numerous types of dendritic cells from various sources have been studied, prepared by a number of techniques. Various means have been developed to use dendritic cells to present the antigen to a tumor site. For example, a number of investigators have reported isolation of dendritic cells, and their use as an adjuvant to enhance an antitumor response. See, e.g., Strome, S. E., et al., “Strategies for Antigen Loading of Dendritic Cells to Enhance the Antitumor Immune Response,” Cancer Res., 62:1884-89 (2002); Mortarini, R. et al., “Autologous Dendritic Cells Derived from CD34⁺ Progenitors and from Monocytes Are Not Functionally Equivalent,” Cancer Res., 57:5534-41 (1997); Toujas, L., “Human Monocyte-Derived Macrophages and Dendritic Cells,” Immunology, 91:635-42 (1997); Chaux, P., et al., “Identification of MAGE-3 Epitopes Presented by HLA-DR Molecules to CD4+ T Lymphocytes,” J. Exp. Med. 189:767-77 (1989); Nestle, F., “Vaccination of Melanoma Patients With Peptide—or Tumor Lysate-pulsed Dendritic Cells,” Nature Med., 4:328-32 (1998); Kotera, Y., “Comparative Analysis of Necrotic and Apoptotic Tumor Cells As a Source of Antigen(s) in Dendritic Cell-based Immunization,” Cancer Res., 61:8105-09 (2001); Kirk, C., et al., “The Dynamics of the T-Cell Antitumor Response,” Cancer Res., 61:8794-8802 (2001); Schnurr, M., “Apoptotic Pancreatic Tumor Cells Are Superior to Cell Lysates,” Cancer Res., 62: 2347-52 (2002).

Regardless of how the dendritic cells are prepared, the key element in their effectiveness is the antigen(s) used to load them. As described earlier, this must be antigen(s) that can trigger clinically effective immune responses against a patient's own tumor. To date, the antigens which have been used to load dendritic cells have been either purified proteins or peptides or non-purified extracts of killed tumor cells or the whole tumor cell itself. As described earlier, all of these antigen sources suffer from problems that limits their effectiveness. Many of these problems can be circumvented by using shed antigens. The use of shed antigens to load dendritic or other types of antigen presenting cells is a strategy that can be applied to enhance the activity of vaccines against all types of cancers, against infectious diseases and against autoimmune diseases. It is therefore an object of the invention to provide a vaccine for cancers, including but not limited to melanoma, breast, pancreatic, colon, lung and brain cancers, as well as viral, bacterial, and other microbiological infectious diseases, and autoimmune diseases using a shed antigen vaccine as set forth, for example, in U.S. Pat. No. 6,338,853 (Bystryn), in an adjuvant of dendritic cells. The entire disclosure of the patents and publications cited herein are incorporated herein by reference.

### SUMMARY OF THE INVENTION

The foregoing and other objects are accomplished, and the disadvantages of earlier attempts are overcome by providing a method for preparing a vaccine suitable for administration to humans for the prevention or treatment of cancer, or for the treatment of infectious or autoimmune diseases which comprises culturing human cancer cells in culture medium; recovering from the culture medium cell surface antigens shed from the cells during culturing; and incubating the recovered shed antigens with dendritic or other types of antigen presenting cells under conditions such that the dendritic cells take up and present to the immune system the shed antigens. The shedding process can be accelerated and enhanced by treating the cells, at an acidic pH, with enzymes or with other agents that promote the release of external cell-surface materials by cells. The vaccine produced from the shed material contains multiple cell surface antigens, including tumor antigens.

The vaccine containing dendritic or other types of cells presenting shed cell surface antigens directed to a particular tumor type may be used for the prevention and/or treatment of cancer in humans by administering the vaccine to a patient several times for one or two months, and then once every one to three months (or less) depending on the particular disease being treated, for an extended period of time. As indicated, the same approach can be used to prepare vaccines to treat or prevent infectious disease caused by viruses (including HIV and oncogenic viruses), bacteria, mycoplasma, fungi, rickettsia, and other cellular and subcellular organisms as well as auto-immune diseases. Alternatively, a shed cell antigen tumor vaccine can be administered concomitantly with dendritic cells to boost immune response as part of antitumor therapy or administered following the use of procedure(s) intended to enhance the number or activation of dendritic or other types of antigen presenting cells in vivo.

### DETAILED DESCRIPTION OF THE PREFERRED EMBODIMENTS

Preparation of Shed Antigen Vaccine

The practice of this invention is hereinafter described with respect to the production of a human melanoma antigen vaccine using dendritic cells or other types of antigen presenting cells as an adjuvant, for the treatment of melanoma patients. As indicated above, however, this invention is also applicable to the production of a human lung cancer vaccine, a human breast cancer vaccine, a human colon cancer vaccine and other human cancer vaccines, as well as vaccines for infectious diseases, particularly infectious diseases caused by bacteria, fungi and other microorganisms, and autoimmune diseases.

A. Vaccine Preparation

We have used the strategy described above to prepare a polyvalent shed antigen vaccine for malignant melanoma. The vaccine was prepared from the material shed into culture medium by a pool of four melanoma cell lines, selected because they express different patterns of cell-surface melanoma-associated antigens. However, other melanoma cells can be used as long as they shed tumor antigens. It is desirable although not necessary that multiple cell lines are used to prepare the vaccine and that the lines are selected based on shedding different but complimentary patterns of tumor antigens so as to increase the repertoire of tumor antigens in the vaccine. It is also desirable but not necessary that the cells be adapted to long-term growth in serum-free medium to exclude these undesirable and highly immunogenic proteins from the vaccine. For vaccine production, the cells were incubated in serum-free and phenol red-free RPMI 1640 medium. After three hours at 37° C., the medium was collected, cells removed by centrifugation at 500×g for 5 min, and cellular debris by a recentrifugation at 2000×g for 10 min. Shed material from the cell lines was concentrated by diafiltration, and the concentrates pooled on an equal protein basis. In some cases, vaccine was prepared with further treatment including the addition of a detergent such as 0.5% Nonidet P-40 (NP-40), followed by ultra-centrifugation at 100,000×g for 90 min, dialysis of the supernatant against normal saline, and passage through a 0.2 um Millex Millipore filter to insure sterility. In all cases the vaccine was adjusted to the desired final protein concentration, vialed, and stored at 70° C. until used. Someone skilled in the art will recognize that different procedures can be used to treat or otherwise purify the shed material to obtain a preparation that may be enriched in a component that is particularly desired or that is more suitable for a particular use and that the shedding process can be accelerated and enhanced by treating the cells with enzymes or other agents that promote the release of external cell-surface material by cells.

1. Antigenic Properties of Vaccine

Shed antigen vaccine prepared from radio iodinated cells was immunophenotyped with a panel of 10 melanoma antisera. The results are summarized in Table 1. Most of the MMs tested were present in the vaccine. Three batches of shed antigen vaccine prepared several months apart all contained the MMs tested, see accompanying Table 1. In more recent studies the vaccine was also shown to contain additional antigens including S100, MAGE-1, MAGE-3, MART-1, gp100, tyrosinase, and TRP-2 which can be detected by their ability to stimulate immune responses in subjects as well as a cytoplasmic antigen described by Dr. Soldano Ferrone.

2. Distribution of MAAs in Various Melanomas

Because it is desirable that the vaccine contain at least one tumor antigen which will be present on most of the melanoma tumors to be treated, the panel of MMs in the vaccine was tested to see if it satisfied this requirement. Fifteen melanomas were lactoperoxidase radio iodinated and immunophenotyped for the MAAs present in the vaccine.

There were marked differences (see Table 3 below) in the pattern of MAAs expressed by each melanoma. However, all of the melanomas expressed several of the MAAs present in the vaccine.

3. Results of Clinical Trials of the Shed, Polyvalent, Melanoma Vaccine

Clinical trials of this vaccine have been conducted in over 600 patients. The vaccine is safe to use as there has been minimal toxicity. Most of the side effects consist of local reactions at the injection site which clear completely in several days. Systemic reactions due to the vaccine occurred in fewer than 10% of patients, and in most cases were mild. This is in contrast to standard therapy of melanoma with interferon alfa-2b, which causes severe toxicity in up to two-thirds of patients.

The vaccine is immunologically active. It stimulates antibody and cellular immune responses against multiple antigens expressed by melanoma. Both types of responses are directed to antigens expressed in vivo by melanoma, indicating they are not directed to artifacts.

The antibody responses can be measured by a variety of techniques including ELISA, Western immunoblotting, and complement dependent cytotoxicity. Using one of these techniques, we found that these antibodies were induced in 51% of 69 sequential patients treated with the vaccine (Oratz, R. et al., “Improved Survival of Melanoma Patients with an Antibody Response to Immunization to a Polyvalent Melanoma Vaccine,” Cancer 75: 495-502,1995). The antibodies were directed to one or more antigens of approximately 45,59,68,79,89,95 and/or 110 kD.

The vaccine also stimulates peptide-specific CD8+ T cells responses against melanoma-associated antigens (Reynolds et al., “Stimulation of CD8+ T Cell Responses to MAGE-3 and MELAN A/MART-1 by Immunization to a Polyvalent Melanoma Vaccine,” Int. J. Cancer, 72: 972-976, 1997; also Reynolds et al., “HLA-independent heterogeneity of CD8+ T cell responses to MAGE-3, Melan A/MART-1, gp100, Tyrosinase, MC1R and TRP-2 in Vaccine—Treated Melanoma Patients,” J. Immunol., 161: 6970-6976, 1998). This is a particularly desirable feature, because CD8+T cells are a major mediator of tumor protective immunity. Vaccine-induced CD8+T responses were detected with a modified and very sensitive ELISPOT assay, described by Reynolds et al., (“Stimulation of CD8+T Cell Responses to MAGE-3 and MELAN A/MART-1 by Immunization to a Polyvalent Melanoma Vaccine,” Int. J. Cancer, 72: 972-976, 1997). Peptide-specific CD8+ T cell responses to MAGE-3 and/or to MART-1 were induced by treatment with the vaccine in 9 (60%) of 15 sequential patients (Reynolds et al., Int J. Cancer, 72: 972-976,1997). In subsequent experiments, responses were also found to be induced against peptides expressed by multiple other melanoma-associated antigens including MAGE-1, gp100, tyrosinase, and TRP-2. The peptides were presented by the HLA class molecules most common among patients with melanoma. These again are desirable features as it does not restrict the use of the vaccine to patients with a particular type of HLA phenotype or whose tumor need to express a particular type of melanoma antigen. Hence, the vaccine can be used to treat a wide spectrum of patients.

The vaccine also stimulates cellular responses that can attack a patient's own melanoma in vivo. This is evidenced by the presence of dense infiltrates of lymphocytes in most (91%) melanoma metastases removed from vaccine-treated patients. Such infiltrates are uncommon in similar nodules removed from non-vaccine-treated patients (Oratz, R. et al., “Induction of Tumor-infiltrating Lymphocytes in Malignant Melanoma Metastases by Immunization to Melanoma Antigen Vaccine,” J. Biol. Res. Modif. 8:355-358,1989).

The vaccine appears clinically effective. In historically controlled trials, we found that the median disease-free and overall survival of vaccine-treated patients (n=94) with resected AJCC stage III melanoma were both 50% longer than that of similar historical controls, ie median recurrance—free survival of 30 months compared to 18 months for historical controls, and overall 5-year survival of 50% vs 33%, respectively (Bystryn, J-C et al., “Relation Between Immune Response to Melanoma Vaccine Immunization and Clinical Outcome in Fstage Ii Malignant Melanoma,” Cancer 69:1157-1164,1992. Also Bystryn, J-C et al., Cancer Vaccines: Clinical Applications: Partially Purified Tumor Antigen Vaccines, in Biologic Therapy of Cancer, 2^{nd} Edition, ed. by V. deVita, S. Hellman and S. A. Rosenberg; J B Lippincott: Philadelphia, pp. 668-679, 1995). The vaccine also appears effective in advanced AJCC stage IV disseminated) melanoma, where the median overall survival of 94 vaccine-treated patients was over 28.6 months compared to 8 months for historical controls. The improvement in outcome for vaccine-treated patients persisted after stratification for site of metastases or tumor load, the strongest predictors of outcome in stage IV melanoma.

As additional evidence of clinical effectiveness, vaccine treatment is associated with a decline in the proportion of patients that have melanoma cells in their circulation. In a study of 118 patients with melanoma, we found that 23% had melanoma cells in their blood (detected by PCR techniques) at baseline prior to vaccine treatment. Three and five months following initiation of vaccine treatment, the proportion of patients with melanoma cells in their blood had declined by 26% and 52% respectively. Furthermore, those patients who had a vaccine-induced decrease in their melanoma cells had a better prognosis that those whose melanoma cells increased, p=0.03 after Cox multi variate analysis (Bystryn, J. C. et al., “Decrease in Circulating Tumor Cells as an Early Marker of Therapy Effectiveness,” in Recent Results in Cancer Research, ed. by Reinhold and Tilgen, Springer-Verlag: Heidelberg, 158:204-207, 2000.

The most compelling evidence that the vaccine is effective is that of a double-blind randomized, placebo-controlled trial conducted with funding from FDA in patients with resected AJCC stage III (disease metastatic to regional nodes) melanoma. The patients were randomly allocated to treatment with the shed, polyvalent melanoma vaccine or with a placebo (normal human albumin) vaccine. Both vaccines were admixed with alum as the adjuvant. Both treatment groups were evenly balanced with respect to prognostic factors. Median length of follow-up was 2.5 years. By Kaplan-Meier analysis, the median recurrence-free survival was two and a half times longer in patients treated with the melanoma vaccine compared to placebo vaccine; i.e., 1.6 years (95% confidence interval 1.0 to 3.0 yrs) vs. 0.6 years (95% confidence interval 0.3 to 1.9 yrs). By Cox proportional hazard analysis this difference was significant: p=0.03. Overall survival was 40% longer in the melanoma vaccine-treated group, i.e., median of 3.8 vs. 2.7 years. To the best of our knowledge, this is the only double-blind trial of a cancer vaccine to have shown a survival advantage for vaccine-treated patients. The results of this trial have been published (Bystryn, J. C. et al., “Double-Blind Trial of a Polyvalent, Shed-antigen, Melanoma Vaccine,” Clin. Cancer Res. 7:1882-1887, 2001).

B. Preparation of Dendritic Cells

Unfortunately, cancer vaccines and many of the newer infectious diseases vaccines are poorly immunogenic. Consequently, a major challenge in the use of vaccines to treat cancer and infectious diseases is to develop immunization procedures that will boost their immunogenicity. Boosting their ability to stimulate cytotoxic, CD 8+ T cell responses is particularly desirable because these cells play a major role in mediating tumor protective immunity.

As described previously, dendritic cells (DC) and other type of antigen presenting cells can strongly increase the immunogenicity of vaccines and particularly their ability to stimulate T cell responses. They do so because they play a critical role in the induction of immune responses. Their role is to pick up and present antigens to immune cells in a manner that will permit the antigen to stimulate these cells to produce antibody and cellular immune responses. They act by ingesting foreign antigens, processing or degrading them into smaller fragments, which are then expressed or presented on the surface of the dendritic cells in association with the major histocompatibility complex (MHC class I or II molecules in mice, or HLA class I or II molecules in humans). Immune cells proliferate and differentiate to produce antibodies or to become cytotoxic T lypohncytes following recognition of specific antigens complexed with the HLA molecules. In some cases, the antigen can bind directly to the class I or II molecule without need for processing within the DC.

Dendritic cells are found in many nonlymphoid tissues but can migrate via the afferent lymph or the blood stream to the T cell-dependent areas of lymphoid organs. They are found in the skin, where they are named Langerhans cells, and are also present in the mucosa. They represent the sentinels of the immune system within the peripheral tissues where they can acquire antigens.

It has been found that loading antigen onto DC can markedly increase the ability of the antigen to stimulate immune responses both in animals and in humans. In fact, the use of DC appears to be one of the most potent procedure to enhance vaccine-induced immune responses.

A wide range of different procedures can be used to enhance vaccine-induced immune responses with DC or other types of antigen presenting cells (Zhou et al., “Current Methods for Loading Dendritic Cells With Tumor Antigen for the Induction of Antitumor Immunity,” Journal of Immunotherapy, 26(4):289-303, 2002). However, all have in common the need to collect the cells, to expand them, to expose them to the antigen(s), and re-administer the cells back to the patients.

A number of variables can affect the effectiveness of the procedure. One of the most important is the nature of the antigen(s) which is used to load the cells. As described above, shed antigens are a superior source of antigens for the production of vaccines against cancer, some infectious diseases, and possibly auto-immune diseases.

Other variables which can affect the effectiveness of the procedure include the source of the dendritic or antigen presenting cells, the manner in which they are treated prior to exposure to the antigen, the manner in which they are loaded with the antigen, and re-administered back to the patients. A variety of additives can be added to the cells during this process to change them in a way which may make them more efficient at ingesting the antigen, processing it, or expressing certain co-factors which improves their ability to stimulate immune cells. In addition, the dendritic cells can be modified to express certain co-factors or immunoenhancing molecules that can enhance their function, or these agents can be co-administered with the antigen loaded dendritic cells. The optimal set of procedures which will be best to generate the dendritic cells, load them with antigen, and re-administer them back to patients may vary with the antigen used or the disease being treated (Zhou, et al.), but can be worked out by persons experienced in the field and may change as the field advances.

1. Collection and Ex Vivo Expansion of Dendritic Cells

Some examples of using DC to enhance vaccine induced immune responses are provided below. Other approaches may be found to work more effectively with a particular type of antigen preparation or for a particular purpose. From the perspective of this invention, the critical element is the use of shed antigens in conjunction with DC or other types of antigen presenting cells.

One procedure for carrying out the process according to the invention for the collection and ex vivo expansion of dendritic cells can be summarized as follows: heparinized blood samples are obtained from the patients. In the process according to the invention, cells which have been isolated from blood can be used as the starting material. This represents a substantial advantage as compared with the process disclosed in EPA 92.400879.0, in which process the cells have to be derived from the bone marrow or umbilical cord blood. Preferably, mononuclear cells (MNC) can be isolated from the apheresis product using suitable separation techniques, in particular by density gradient centrifugation through FICOLL (a neutral, highly branched, hydrophilic polymer of sucrose (Pharmacia, New Jersey).

Another alternative procedure for ex vivo expansion of hematopoietic stem and progenitor cells is described in U.S. Pat. No. 5,199,942, incorporated herein by reference. Other suitable methods are known in the art. Once collected and isolated, DC or other types of antigen presenting cells are normally expended, matured and activated by incubation with a variety of cellular growth factors as described in U.S. Pat. No. 5,199,942. Other factors such as flt3-L, IL-1, IL-3 and c-kit ligand, can be used. Alternatively, cytokines may be administered prior to, or concurrently with the collection of blood mononuclear cells to expend the population of DC ands DC progenitor cells.

The dendritic cells or antigen presenting cells which are obtained in this way can be subjected to further treatment, depending on the purpose, and then reintroduced into the patient, or used to make antigen activated vaccine, wherein the dendritic cell acts as an Antigen Presenting Cell or APC. A leucapheresis is particularly helpful when relatively large quantities of dendritic cells are required. The mononuclear cells are subjected to further treatment in order to enrich those cells which possess desirable properties. The dendritic cells described herein are then used for vaccine development.

Once expanded, dendritic cells are then pulsed with (exposed to) antigen, to allow them to take up the antigen in a manner suitable for presentation to other cells of the immune system. The various procedures that can be used are described in Zhou et al. Antigens are classically processed and presented through two pathways. Peptides derived from proteins in the cytosolic compartment are presented in the context of Class I MHC molecules, whereas peptides derived from proteins that are found in the endocytic pathway are presented in the context of Class II MHC. However, those of skill in the art recognize that there are exceptions; for example, the response of CD8⁺ tumor specific T cells, which recognize exogenous tumor antigens expressed on MHC Class I. A review of MHC-dependent antigen processing and peptide presentation is found in Germain, R. N., Cell 76:287 (1994).

Numerous methods of pulsing dendritic cells with antigen are known (see Zhou et al.); those of skill in the art regard development of suitable methods for a selected antigen as routine experimentation. In general, the antigen is added to cultured dendritic cells under conditions promoting the phagocytic capacity, maturation and activation of these cells, and the cells are then allowed sufficient time to take up and process the antigen, and express antigen peptides on the cell surface in association with either Class I or Class II MHC, and mature and become activated a period of about 24 hours (from about 3 to about 30 hours, preferably 4-6 hours).

The principles of this invention can also be applied to prepare improved vaccines for infectious and for autoimmune diseases. For example, dendritic cells can be exposed to a desired cancer antigen or antigenic composition by incubating the dendritic cells with the antigen in vitro in culture medium. In one mode, the antigen in aqueous soluble or aqueous suspension form, is added to cell culture medium at the same time as the dendritic cells. The dendritic cells advantageously take up antigen for successful presentation to T cells. In another mode, antigens are introduced to the cytosol of the dendritic cells by alternate methods, including but not limited to osmotic lysis of pinocytic vesicles, the use of pH, or antigen coated or loaded liposomes or other types of small particles (“Introduction of Macromolecules Into Cultured Mammalian Cell by Osmotic Lysis of Pinocytic Vesicles,” Cell 29:33; Poste et al., “Lipid Vesicles as Carriers for Introducing Biologically Active Materials Into Cells,” Methods Cell Biol. 14:33(1976); Reddy et al., “pH Sensitive Liposomes Provide an Efficient Means of Sensitizing Target Cells to Class I Restricted CTL Recognition of a Soluble Protein,” J. Immunol. Methods 141:157 (1991), Zhou et al.).

C. Administration of Activated, Antigen-Pulsed Dendritic Cell

The present invention provides methods of forming cancer vaccines comprising shed antigen vaccine with an activated, antigen-pulsed dendritic cell adjuvant. The use of such cells in conjunction with cytokines, or other immunoregulatory molecules that can enhance the activity of dendritic or other antigen presenting cells is also contemplated. The inventive compositions are administered to stimulate an immune response, and can be given by bolus injection, continuous infusion, sustained release from implants, or other suitable technique. Typically, the improved vaccine of the present invention will be administered in the form of a composition comprising the shed antigen-pulsed, dendritic cells in conjunction with physiologically acceptable carriers, excipients or diluents. Such carriers will be nontoxic to recipients at the dosages and concentrations employed. Neutral buffered saline or saline mixed with conspecific serum albumin are exemplary appropriate diluents.

For use in stimulating a certain type of immune response, the improved vaccine can be administered along with other cytokines or immunomodulatory agents, which improve the immune response. Several useful cytokines (or peptide regulatory factors) are discussed in Schrader, J. W. (Mol. Immunol. 28: 295; 1991). Such factors include (alone or in combination) Interleukins 1,2,4,5,6,7,10,12 and 15; granulocyte-macrophage colony stimulating factor, granulocyte colony stimulating factor; a fusion protein comprising Interleukin-3 and granulocyte-macrophage colony stimulating factor; Interferon-γ, TNF, TGF-β, flt-3 ligand and biologically active derivatives thereof. A particularly preferred cytokine is CD40 ligand (CD40L). A soluble form of CD40L is described in U.S. Pat. No. 5,962,406 (Armitage). Other cytokines will also be useful, as described herein. DNA or RNA encoding such cytokines will also be useful in the inventive methods, for example, by transfecting the dendritic cells to express the cytokines. Administration of these immunomodulatory molecules includes simultaneous, separate or sequential administration with the antigen-pulsed dendritic cells of the present invention.

TABLE 1
MAA immunophenotyping of melanoma vaccine

| | |
|---|---|
| MAA | Presence of MAA |
| defined | (vaccine batch) |

| Antisera | (kilodaltons) | 1 | 2 | 3 | Ref. |
|---|---|---|---|---|---|
| Mouse monoclonal | | | | | |
| 225.28S | 240+ | + | + | + | 23 |
| 9.2.27 | 240+ | + | + | + | 24 |
| 436.G10 | 122-130 | 0 | NT^{b} | NT | |
| Nu4B | 26, 29, 95, 116 | + | NT | NT | 25 |
| 376.96 | 94 | 0 | NT | NT | 17 |
| 118.1 | 94-97 | + | + | + | 15 |
| 465.12S | 94 | 0 | NT | NT | 27 |
| MeTBT | 69-70 | 0 | NT | NT | 26 |
| Rabbit polyclonal | | | | | |
| SB29, SB54 | 240 | + | + | + | 11 |
| SB29, SB54 | 150^{a} | + | + | + | 11 |
| SB29, SB54 | 140^{a} | + | + | + | 11 |
| SB29, SB54 | 120 | + | + | + | 11 |
| SB29, SB54 | 95 | + | + | + | 11 |
| SB29, SB54 | 75 | + | + | + | 11 |

^{a}Not reactive with SB54.
^{b}(NT) not tested.

TABLE 2
Effect of detergent and ultracentrifugation on macromolecules,
MAAs, and Dr antigens in material shed by melanoma cells

| | Presence in shed material after ultracentrifugation | | | | | |
|---|---|---|---|---|---|---|
| | ¹²⁵I- | | | | | |
| | macromolecules^{b} | | ¹²⁵I-MAAs^{c} | | ¹²⁵I-Dr^{c} | |
| | | Change^{d} | | Change | | Change |
| Treatment^{a} | cpm | (%) | cpm | (%) | cpm | (%) |
| None | 10,362 | | 817 | | 428 | |
| Ultra- | 6,325 | −40 | 258 | −70 | 0 | −100 |
| centrifugation | | | | | | |
| NP-40 + ultra- | 8,612 | −17 | 574 | −30 | 0 | −100 |
| centrifugation | | | | | | |

^{a}Material shed by radioiodinated melanoma cells was ultracentrifuged in 0.5 ml aliquots at 100,000 g for 90 min, incubated in a final concentration of 0.5% NP-40 for 2 h prior to ultracentrifugation, or not treated. All were subsequently assayed for radioactivity associated with macromolecules, MAAs defined by antiserum SB29, or Dr antigens. All assays were performed on 0.025 ml aliquots of material in the presence of a final concentration of 0.5% NP-40.
^{b}Assayed by precipitation with 10% trichloroacetic acid.
^{c}Assayed by protein A-immune precipitation with specific antisera.
^{d}From untreated control.

TABLE 3
Surface MAAs expressed by melanomas in various individuals

| | | Expression of MAA in melanoma | | | | | | | | | | | | | | |
|---|---|---|---|---|---|---|---|---|---|---|---|---|---|---|---|---|
| MAA | Antiserum | HM31 | HM34 | HM49 | HM54 | HM60 | HM80 | G361 | SK23 | SK27 | SK28 | SK29 | SK37 | M14 | M20 | VA1 |
| 240- | SB29 | + | − | − | ++ | +++ | − | +++ | ++ | ++ | ++ | + | − | ++ | ++ | ++ |
| | SB54 | + | − | − | + | − | − | ++ | − | − | − | − | − | − | − | − |
| | 225.28S | − | − | − | − | +++ | +++ | − | + | − | +++ | +++ | +++ | +++ | +++ | − |
| | 9 2 27 | − | − | − | − | +++ | +++ | − | + | ± | +++ | +++ | +++ | +++ | +++ | − |
| 150 | SB29 | + | + | + | + | − | − | + | − | − | − | − | − | ++ | − | − |
| 140 | SB29 | ++ | − | + | +++ | − | − | +++ | − | − | − | − | − | − | − | − |
| 120 | SB29 | +++ | ++ | + | +++ | − | − | +++ | − | − | − | − | − | − | − | − |
| | SB54 | ++ | + | + | ++ | − | − | ++ | − | − | − | − | − | − | − | − |
| 116 | Nu4B | − | − | | − | − | − | − | − | − | − | + | − | − | + | − |
| 95-97 | SB29 | ++ | + | − | +++ | + | − | +++ | + | + | − | − | − | − | − | + |
| | SB54 | ++ | + | − | +++ | − | − | +++ | − | − | − | − | − | − | − | − |
| | 118.1 | − | − | − | − | ++ | +++ | − | +++ | +++ | +++ | − | +++ | ++ | +++ | +++ |
| 75 | SB29 | ++ | − | − | +++ | +++ | +++ | +++ | +++ | +++ | +++ | ++ | + | ++ | ++ | +++ |
| | SB54 | + | − | − | + | − | − | + | + | − | − | − | − | − | − | − |
| 70 | Me3 TBT | − | − | | − | − | − | − | − | − | | − | − | − | − | − |

^{a}Assayed by indirect immunoprecipitation with protein A-sepharose.

TABLE 4
Characteristics of immunized patients

| | | | | | Duration of | | | |
|---|---|---|---|---|---|---|---|---|
| | | | Previous | | metastatic | | | |
| | | | treatment | | disease prior | | | Length of |
| Patient | | | other than | Site of | to immunization | No of | Current | follow-up^{b} |
| no | Age | Sex | surgery | metastasis | (months) | immunization | status^{a} | (months) |
| 1 | 31 | F | BCG. DTIC | Skin. lung | 12 | 10 | P | ¼ |
| 2 | 24 | F | None | Lung | 2 | 10 | P | 3 |
| 3 | 53 | M | None | Skin | 2 | 13 | | 8 |
| 4 | 58 | F | None | Skin | 2 | 8 | P | 1 |
| 5 | 48 | M | None | Skin | 1 | 12 | P | 3 |
| 6 | 54 | M | None | Skin | 2 | 11 | P | 3 |
| 7 | 58 | M | None | Skin | 2 | 14 | P | 4 |
| 8 | 68 | M | None | Skin. lung | 2 | 10 | P | 6 |
| 9 | 75 | F | DTIC. | Skin | 36 | 17 | S | 14 |
| | | | Actinomycin D | | | | | |
| 10 | 46 | M | None | Skin | 1 | 18 | R | 24 |
| 15 | 29 | M | None | Skin | 4 | 8 | P | 2 |
| 17 | 68 | M | None | Skin | 4 | 8 | P | 2 |
| 20 | 38 | M | None | Skin. lung | 3 | 13 | P | 4 |

^{a}(P) progression. (S) stable: (R) regression
^{b}From onset of immunotherapy
^{c}(BCG) bacillus Calmette-Guerin

TABLE 5
Immunogenicity of melanoma vaccine

| Patient | Immune response to melanoma^{a} | | |
|---|---|---|---|
| no. | Humoral^{b} | Cellular^{c} | Either |
| 1 | ++ | 0 | 5 |
| 2 | + | 10 | + |
| 3 | + | 0 | + |
| 4 | 0 | 0 | 0 |
| 5 | ± | 5 | 0 |
| 6 | 0 | 0 | 0 |
| 7 | 0 | 0 | + |
| 8 | 0 | 0 | 0 |
| 9 | 0 | 20 | + |
| 10 | + | 10 | + |
| 15 | ++ | 0 | + |
| 17 | 0 | NT | 0 |
| 20 | NT | 25 | + |
| No. (%) positive: | 5 (38%) | 4 (31%) | 8 (62%) |

^{a}(±) 25-49%, (+) 50-100%, and (++) 100%, increase over preimmunization level of melanoma antibodies; (NT) not tested.
^{b}By indirect immunoprecipitation of ¹²⁵I-melanoma macromolecules.
^{c}By skin test to 10 μg vaccine; results represent millimeters of average induration at 24-48 h.

TABLE 6
Antibodies to fetal calf serum proteins
in patients immunized to melanoma vaccine

| | | ¹²⁵I-FCS^{a} |
|---|---|---|
| | Antibodies | (2 months |
| Patient no. | to preimmunization | postimmunization) |
| Melanoma | | |
| 1 | 18.3 | 0.7 |
| 2 | 0.0 | 0.1 |
| 3 | 0.0 | 0.0 |
| 4 | 0.1 | 0.1 |
| 5 | 0.1 | 0.2 |
| 6 | 0.1 | <0.1 |
| 7 | <0.1 | 0.1 |
| 8 | <0.1 | <0.1 |
| 9 | 0.6 | 0.8 |
| 10 | 0.0 | 0.0 |
| 15 | 0.1 | <0.1 |
| 17 | 0.3 | 0.4 |
| 20 | 0.0 | 0.0 |
| Normal | | |
| 2003 | 0.0 | |
| 2004 | 0.1 | |
| 2005 | <0.1 | |
| 2006 | 0.0 | |
| 2007 | 0.0 | |
| 2008 | 0.0 | |
| 2009 | 0.0 | |
| 2010 | 0.1 | |
| 2011 | 0.0 | |
| 2012 | 0.0 | |
| 2013 | 0.0 | |
| ANTI-FCS | 68.0 | |

^{a}Percent of radioactivity associated with ¹²⁵I-FCS specifically immunoprecipitated by serum and protein A-sepharose.

I claim:

1. A method for producing a composition for use as a vaccine for treatment or prevention of cancer, comprising:
a. collecting antigens released or shed by the type of tumor cell against which it is desired to prepare the vaccine;
b. preparing mammalian dendritic cells in a culture from a mammalian blood, bone marrow or other tissue sample by culturing the blood, bone marrow, or other tissue sample under conditions that cause differentiation and proliferation of dendritic cells;
c. separating dendritic cells from other cells in the culture; and
d. exposing the dendritic cells to the shed antigens collected as described in paragraph a. above under conditions that result in the combination of the shed cancer antigens or their fragments and the dendritic cells.

2. A method in accordance with claim 1, wherein the blood, bone marrow or other tissue sample is taken from the patient receiving the treatment or from an unrelated donor.

3. A method in accordance with claim 1, wherein the shed cancer antigens are obtained from one or more melanoma cell lines.

4. A method in accordance with claim 1 wherein the shed mammalian cancer antigens are obtained from one or more breast cancer cell lines.

5. A method in accordance with claim 1 wherein the shed mammalian cancer antigens are obtained from one or more lung cancer cell lines.

6. A method in accordance with claim 1 wherein the shed mammalian cancer antigens are obtained from one or more prostate cancer cell lines.

7. A method in accordance with claim 1 wherein the shed mammalian cancer antigens are obtained from one or more colon cancer cell lines.

8. A method in accordance with claim 1 wherein the shed mammalian cancer antigens are obtained from one or more ovarian cancer cell lines.

9. A method in accordance with claim 1 wherein the shed mammalian cancer antigens are obtained from one or more cancer cell lines of other histological type.

10. A method in accordance with claim 1 wherein the shed antigens are obtained from one or more pathogenic strain of bacteria, mycobacteria, fungi, virus, or other pathogenic organism.

11. A method in accordance with claim 1 wherein the shed antigens are obtained from one or more normal cell lines to treat an auto-immune disease.

12. A method in accordance with claim 1 wherein the shed cancer, infectious organism or normal tissue antigens are loaded onto antigen presenting cells including macrophages, Langerhan's cells, or other types of antigen presenting cells.

13. A method in accordance with claim 1 wherein the shed antigen vaccine loaded onto dendritic or other type of antigen presenting cell is co-administered with immunomodulators that can upregulate vaccine-induced immune responses such as IL-2 or GM-CSF.

14. A method in accordance with claim 12 wherein the shed antigen vaccine loaded onto dendritic or other type of antigen presenting cells is co-administered with immunomodulators that can upregulate vaccine-induced immune responses such as IL-2 or GM-CSF. (Same as claim 13, but dependent on claim 12)

15. A method in accordance with claim 1 wherein the shed antigens are collected from several different lines of tumor cells which shed different but complimentary patterns of tumor antigens so as to broaden the spectrum of tumor antigens in the vaccine preparation.

16. A method in accordance with claim 1 wherein the cells:
a. are adapted to long-term growth in serum-free medium; and
b. are treated at an acid pH, or with certain enzymes or other agents which accelerate or enhance the release of material from the cell-surface.

17. A method for treating tumor in a patient comprising administering an effective an effective amount of a vaccine made in accordance with claim 1.

18. A method in accordance for treating cancer comprising administering an effective amount of a vaccine produced in accordance with claim 1.

19. A method for producing an immune response in a patient comprising administering an effective amount of a vaccine made in accordance with the method of claim 1.

20. A method in accordance with claim 19, wherein dendritic cells present shed tumor antigens to the immune system with dendritic cells.

21. A vaccine for treating cancer in a patient, comprising a composition made in accordance with the method of claim 1 in a pharmaceutically acceptable vehicle.
